(19) United States
(12) Patent Application Publication
(10) Pub. No.: US 2012/0213874 A1
(43) Pub. Date: Aug. 23, 2012

(54) HERBAL SOLID FORMULATION AND PROCESS FOR PREPARING THE SAME

(75) Inventors: Shankar Kumar MITRA, Bangalore (IN); Uddagiri Venkanna BABU, Bangalore (IN); Ekta SAXENA, Bangalore (IN); Anu Vrat SHARMA, Bangalore (IN)

(73) Assignee: HIMALAYA GLOBAL HOLDINGS LTD, Grand Cayman (KY)

(21) Appl. No.: 13/011,731

(22) Filed: Jan. 21, 2011

Related U.S. Application Data
(63) Continuation-in-part of application No. 13/003,543, filed on Aug. 1, 2011.

(51) Int. Cl. A61K 36/906; A61K 36/328; A61K 36/18

(52) U.S. Cl. 424/756; 424/725; 424/779

(57) ABSTRACT

Disclosed is a herbal solid formulation comprising Withania somnifera, Zingiber officinale or Commiphora mukul extracts essentially free of excipients and preservatives and process for preparing the same.

This application is a continuation-in-part of U.S. application Ser. No. 13/003,543 which is a U.S. national stage entry of PCT application number PCT/IB2008/001797 filed Jul. 9, 2008 the entire disclosures of which are expressly incorporated by reference herein.

### FIELD OF THE INVENTION

This invention, in general relates to a herbal solid formulation. In particular, the present invention provides a herbal solid formulation comprising Withania somnifera, Zingiber officinale or Commiphora mukul extracts without using any excipients and preservatives and process for preparing the same.

1. Background of the Invention

Herbal supplements have witnessed tremendous growth and acceptance among the consumers during the last decade due to their safety and efficacy. Unlike allopathic medications, herbal extracts are safe and devoid of any side effects. There is a growing concern among the consumers worldwide using naturally derived products and avoiding synthetic chemicals in their food, personal care products and daily health supplements. Many herbal products that are available in the market as tablets and capsules use synthetic excipients such as binders, lubricants, diluents and preservatives such as parabens and salts of benzoic acids etc. These excipients and preservatives are reported to have toxic and side effects.

Pharmaceutical dosage forms such as tablets and capsules should have certain properties such as hardness, friability, disintegration time (DT), stability and delivery of the drug to give required therapeutic benefits to the patient. These properties are achieved using the excipients such as binders, lubricants and diluents.

It is therefore very important and challenging task to develop a process of manufacturing herbal solid formulation without using any synthetic excipient and preservative.

2. Related Art

U.S. Pat. No. 6,207,189 by Mercati et al disclose a process for the production of tablets and capsules of natural substances of vegetable origin wherein dry extracts and micronized powders of one or more medicinal herbs in appropriate proportions are blended and subjected to steam pressure followed by drying, preparation of granules and compression to tablets.

U.S. Pat. No. 6,468,563 by Schmidt et al. discloses a process for producing rapidly disintegrating pharmaceutical formulation containing an extract and lubricant and compressing the blend to form the pharmaceutical formulation.

### SUMMARY OF THE INVENTION

It is a principal object of the invention to provide a herbal solid formulation of comprising Withania somnifera, Zingiber officinale or Commiphora mukul extracts essentially free of additives/excipients and preservatives and providing required quantity of active constituents per dose.

It is another object of the invention to provide a herbal solid formulation of herb comprising Withania somnifera, Zingiber officinale or Commiphora mukul extracts having reduced side effects and toxicity.

It is yet another object of the invention to provide a herbal solid formulation of herb comprising Withania somnifera, Zingiber officinale or Commiphora mukul extracts essentially free of excipients/additives and preservatives and having desired friability, disintegration time and hardness.

It is still another object of the invention to provide a method for preparing extract of herb comprising Withania somnifera, Zingiber officinale or Commiphora mukul used to prepare the solid formulation.

The above and other objects of the present invention are attained according to following preferred embodiments of the present invention. However the scope of the invention is not restricted to the particular embodiments discussed herein after.

In accordance with one preferred embodiment of the present invention, there is provided a herbal solid formulation comprises a blend of Super Critical Fluid (CO₂) extract, water extract and powder of Withania somnifera, Zingiber officinale or Commiphora mukul, wherein said herbal solid formulation is essentially free of additives/excipients.

In accordance with one preferred embodiment of the present invention, there is provided a herbal solid formulation comprises a blend of Super Critical Fluid (CO₂) extract, water extract and powder of Withania somnifera, Zingiber officinale or Commiphora mukul, wherein said blend of extract and said powder of herb is mixed in a ratio of about 1:0.5 to about 1:10.

In accordance with another preferred embodiment of the invention there is provided a process for preparing a herbal solid formulation essentially free of additives/excipients comprising granulating a blend of Super Critical Fluid (CO₂) extract and water extract of Withania somnifera, Zingiber officinale or Commiphora mukul autoclaving the resultant granular blend and further lubricating the granulated blend by adding the powder extract of Withania somnifera, Zingiber officinale or Commiphora mukul and preparing the solid formulation.

In accordance with yet another preferred embodiment of the invention, the powder of herb is obtained by pulverizing the herb to a powder having mesh size preferably between about 10 to about 100, more preferably between 20 to 80.

In accordance with still another embodiment of the present invention, wherein the granulation of the blend of extracts and powder of the herb is carried out in presence of a solvent, preferably water and grain alcohol or combination thereof.

In accordance with yet another embodiment of the present invention, there is provided a process for preparing the extract of the herb by Super Critical Fluid (CO₂) extraction, percolation, hot soxhalation or refluxing followed by filtration and concentration to dryness at optimum temperature.

In accordance with one other embodiment of the present invention, there is provided a process for sterilization of herbal powders by autoclaving the granular mixture, wherein autoclaving prevents microbial growth.

### BRIEF DESCRIPTION OF DRAWINGS

Further objects of the present invention together with additional features contributing thereto and advantages accruing there from will be apparent from the following description of preferred embodiments of the invention which are shown in the accompanying drawing figures, wherein:

FIG. 1 illustrates the LCMS chromatogram of Withania somnifera CO₂ extract.

FIG. 2 illustrates the LCMS chromatogram of Ginger (Zingiber officinale) CO₂ extract.

FIG. 3 illustrates the LCMS chromatogram of Ginger (Zingiber officinale) CO₂ extract.

FIG. 4 illustrates the LCMS chromatogram of Guggul CO₂ extract.

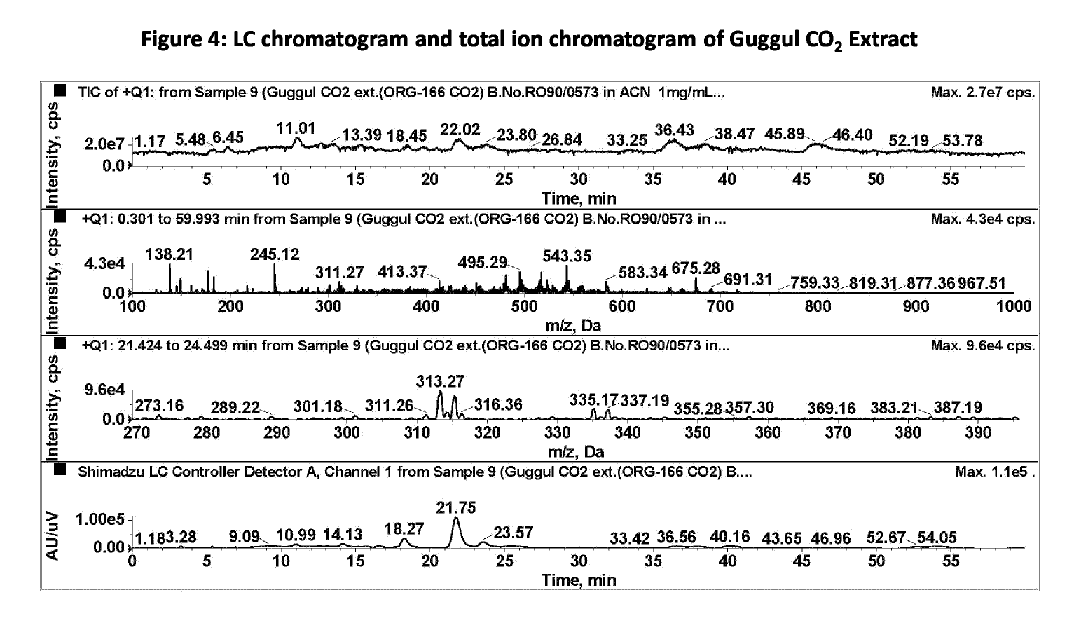

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The present invention provides a herbal solid formulation essentially free of excipients/additives or preservatives, wherein said formulation comprises a blend of Super Critical Fluid (CO₂) extract, water extract of Withania somnifera and powder Withania somnifera, Zingiber officinale or Commiphora mukul and a process for preparing the same.

The process of preparing the herbal solid formulation involves granulation of the blend of extracts and powder of the herb using a solvent system and autoclaving the granules. The solvent system employed for granulating the mixture includes grain alcohol, water or combination thereof. Autoclaving helps in microbial control of the solid formulation as it does not contain any preservatives.

The autoclaved granules are further lubricated using the powder of the Withania somnifera, Zingiber officinale or Commiphora mukul and compressed or encapsulated into tablets or capsules.

The extracts of the herb is prepared by Super Critical Fluid (CO₂) method also the same is prepared by employing percolation, hot soxhalation or refluxing method using a solvent, followed by filtration and concentration on a rotatory evaporator on steam bath at optimum temperature and under reduced pressure. The solvent employed includes organic grain alcohol, ethanol or water or combinations thereof, preferably grain alcohol.

The powder of the herb is prepared by pulverizing the root of herb to a powder of different mesh sizes based on the requirement, preferably between about 10 to about 100, more preferably between 20 to 80. The extract and the powder of the herb is mixed in a predetermined ratio preferably between about 1:0.5 to about 1:10 for optimum granulation.

All extracts, granules and tablets are subjected to standardization by High Performance Thin Layer Chromatography (HPTLC) and High Performance Liquid Chromatography (HPLC) for identification and quantitative estimation of active marker compounds. The extracts were evaluated for toxicity studies in rats and tablets for safety studies in healthy human volunteers.

The solid formulation according to the present invention has desired hardness preferably between about 3 to about 4 kg/cm², friability less than about 1% and disintegration time less than about 30 min. The solid formulation complies with USFDA guidelines.

According to the present invention, the disclosed solid formulation is preferably granules, tablet or capsule.

The following non-limiting examples illustrate specific embodiments of the present invention. They are, not intended to be limiting the scope of present invention in any way.

### EXAMPLE-1

Preparation of Withania somnifera Water Extract

Approximately 100 Kg of shade dried plant material was subjected to extraction with 400 litres of purified water by percolation method at room temperature. The water extractions after 24-48 hours were filtered through muslin cloth and concentrated to thick paste. After achieving the desired total solid content, the soft extract was spray dried to a free flowing dry extract powder. The water extract was also prepared by hot soxhalation method.

### EXAMPLE-2

Preparation of Withania somnifera SCFE (CO₂) Extract

Approximately 25 Kg of the roots of Withania somnifera was pulverized to fine powder and loaded in to extractor. Super Critical Carbon dioxide liquid was pumped into the extractor at a pressure of 300 bar and 39° C. temperature for 2-3 hours. Extract was separated into the container at pressure of 40 bar and 20° C temperature. The CO₂ super critical liquid was recycled from the extraction vessel. The resultant extract was analyzed for active markers of Withania somnifera.

### EXAMPLE-3

### Preparation of Organic Withania Granules (Formula-1)

TABLE 1

| S. | | Weight per | Weight per |
|---|---|---|---|
| No. | Name of the Material | Tablet in mg | Batch in kg |
| 1 | Withania somnifera (water extract) | 280.00 | 28.00 |
| | (#40 mesh) | | |
| 2 | Withania somnifera (supercritical | 10.00 | 1.00 |
| | fluid extract, CO₂) | | |
| 3 | Withania somnifera roots | 380.00 | 38.00 |
| | parts (# 80 mesh) | | |

### EXAMPLE-4

### Formula of Lubrication of Granules

TABLE 2

| S. | | Weight per | Weight per |
|---|---|---|---|
| No. | Name of the material | Tablet in mg | Batch in Kg |
| 1 | Withania somnifera Extract Granules | 670 | 67.00 |
| | (# 16 mesh) | | |
| 2 | Withania somnifera (#40 mesh) | 0 | 0 |
| | powder | | |
| | Total | 670 | 67.00 |

### EXAMPLE-5

### Manufacturing Procedure of Granulation and Compression

Dispensing and Sifting of Raw Materials and Extracts:
1. Dispensed the raw materials as per Batch Formula.
2. Transferred 38.00 Kg roots plant powder into trays and sterilized the material@160° C. for 2 hours.
3. Unloaded the autoclaved materials into double lined polybags separately and kept in airtight containers.
4. Sent the sample for LOD, BD and Microbiological Analysis.
5. If required Autoclaved herbal material at 121° C. for 40 min.
6. Sifted 38.00 kg of herbal root powder through #80 sieve.
7. Sifted 28.00 kg of water extract through #40 sieve.
8. Collected the above-sifted materials in separate duly labeled double lined polybags.
9. Recorded Quantity sifted and the sieve integrity before and after sifting.

### Granulation

1. Charged 38.00 Kg of root powder and 1.0 Kg of roots CO₂ extract into the RMG mix for about 5 minute.
2. Added 28.00 Kg of water extract and mixed for another 5 min.
3. Added Purified water to the RMG containing the root powder, roots CO₂ extract and water extract and mixed over a period of about 3 minute, with a medium speed.
4. Stopped the mixer and scraped off the mass from the sides and bottom.
5. Continued mixing by operating the impeller at high speed with Chopper ON for about 2 minute.
6. Added additional quantity of Purified water, if required.
7. Discharged the mass from the RMG.

### Wet Milling

1. Milled the Wet Mass obtained in Multi mill fitted with 8 mm screen.

### Drying

1. Dried the Wet mass obtained in Tray Drier/FBD at about 55° C. to 65° C. for about 60 minutes.
2. Checked the Moisture once every 30 minutes (LOD Limit: 3.0 to 4.0% w/w) and recorded the details.

### Sizing

1. Sifted the dried granules using a Sifter fitted with #16 sieve.
2. Collected the sifted granules in a clean double poly-lined HDPE container.
3. Milled the retains (oversize granules) obtained through a Multi Mill fitted with 1.5 mm screen with ‘Knives Forward’ direction.
4. Passed the milled granules obtained through a Sifter fitted with #16 sieve.
5. Collected the sized granules obtained and added them to the sifted granules obtained in stage 10.3.
6. Blended the above sifted granules for about 3 minutes at 20-25 RPM.
7. Unloaded the blend in a clean double poly-lined HDPE drums and affixed duly filled status labels.
8. Weighed the blend and entered the details.

TABLE 3

| Parameter | Standard value |
|---|---|
| Loss on drying | 3.0-4.0% w/w |
| Bulk density | 0.45-0.65 g/ml |
| Granules to fine ratio | 60:40-90:10 |
| Actives | As per Finished Product spec |
| TVAC | NMT 10000 cfu/gm |
| Fungal count | NMT 100 cfu/gm |

### Compression

Adjust the machine as below mentioned tooling in the table

TABLE 4

| | Description |
|---|---|
| Punch Size | 15 × 7 mm Caplet |
| Upper Punch | Plain |
| Lower Punch | Plain |

1. Carried out the initial checks before starting the operation as specified in standard parameters.
2. Checked for Appearance, Average Weight, Individual weight, Thickness, Hardness, Friability & DT of 6 tablets, checked Appearance, Thickness & Hardness of tablets every 30 min, Checked Friability and DT every 1-hour.
3. Checked for the Average weight of 20 tablets every 30 mins graphically.
4. Collected the tablets in a double poly-lined, tightly closed, container. Weighed each container and entered the details.
5. Collected the Compressed tablets in Double poly-lined HDPE drums and recorded their weights.

### EXAMPLE-6

Finished Product Specification of Withania somnifera Per Caplet

TABLE 5
STANDARD PARAMETERS

| | |
|---|---|
| 1. Theoretical average | 670 mg |
| 2. Weight uniformity | 670 mg ± 5% (636.5 to 703.5 mg) |
| 3. Weight of 20 tablets | 13.40 g ± 5% (12.73 to 14.07 gm) |
| 4. Tablet thickness | 5.8 to 6.8 mm |
| 5. Tablet hardness | 2 to 6 Kg/cm² |
| 6. Friability | NMT 1.0% W/W |
| 7. Disintegration time | NMT 30 min. |
| 8. Total withanolide content | 2.96 mg-3.91 mg |

### EXAMPLE-7

Estimation of Amino Acids of Water Extracts of Withania somnifera Roots by Amino Acid Analyzer.

The samples were analyzed by test method “J. AOAC (Journal of Association of Official Agricultural Chemists), 70, 241-247, 1987”. The results are provided in below table;

TABLE 6

| S. No | Amino Acids Composition | % |
|---|---|---|
| 1 | Aspargine | 0.39 |
| 2 | Glutamine | 1.49 |
| 3 | Hydroxy proline | 0.29 |
| 4 | Serine | 0.21 |
| 5 | Glycine | 0.45 |
| 6 | Histidine | 0.09 |
| 7 | Arginine | 0.02 |
| 8 | Threonine | 0.51 |
| 9 | Alanine | 0.23 |
| 10 | Proline | 0.59 |
| 11 | Tyrosine | 0.03 |
| 12 | Valine | 0.22 |
| 13 | Methionine | 0.63 |
| 14 | Isoleucine | 0.13 |
| 15 | Leucine | 0.17 |
| 16 | Phenyl alanine | 0.11 |
| 17 | Lysine | 0.03 |

### EXAMPLE-8

Estimation of total Withanolides in Withania somnifera.

Reference method for analysis: Standardisation of botanicals (Testing and extraction methods of medicinal herbs)—By Dr. V. Rajpal, Volume 1, page-256. Eastern publishers, New Delhi, year 2002.

Withanolides can be extracted by methanol and the ether soluble portion of methanolic extract is taken. The ether soluble portion contains total withanolides, which is dried and weighed.

Estimation procedure for dry extracts and granules:
1. Weigh about 5 g (W) of finely powdered Withania somnifera dry extract/granules in 100 ml or 250 ml beaker.
2. Measure 25 ml of methanol and 25 ml of water in a beaker and add to it.
3. Stir for 15 minutes using a magnetic stirrer at room temperature. Filter through ordinary filter paper without loosing the residue. Extract the residue in the similar manner with 3×50 ml of methanol: water (1:1) mixture and filter the aqueous methanol extract.
4. Combine the aqueous methanol extracts and transfer to a 500 ml separating funnel.
5. De-fat the extract by gentle shaking with 100 ml of hexane. Discard the hexane layer and repeat the de-fatting two more times with 100 ml each of hexane and discard the hexane layers.
6. Fractionate the remaining aqueous methanolic extract by gentle shaking for 2 minutes with 100 ml of Diethyl ether.
7. Repeat the fractionation with 2×100 ml of diethyl ether using the separating funnel.
8. Combine the ether extracts, wash with 2×50 ml of water using separating funnel. Separate the water layer and discard.
9. Dry this ether fraction over a pre-weighed (W₁) china dish on an evaporation water bath maintained at about 60° C. Keep the dried china dish in oven for 30 minutes at 105° C., Cool and weigh the residue in china dish (W₂).
10. The residue represents the totalwithanolides, which can be expressed as % w/w as the original material by calculating total withanolides using formula.

### Calculation for Withanolides:

$$\% w / w of withanolides = \frac{Weight of the residue ( {W}_{2} - {W}_{1} ) in g}{Weight of the sample taken ( W ) in g} \times 100$$

### EXAMPLE-9

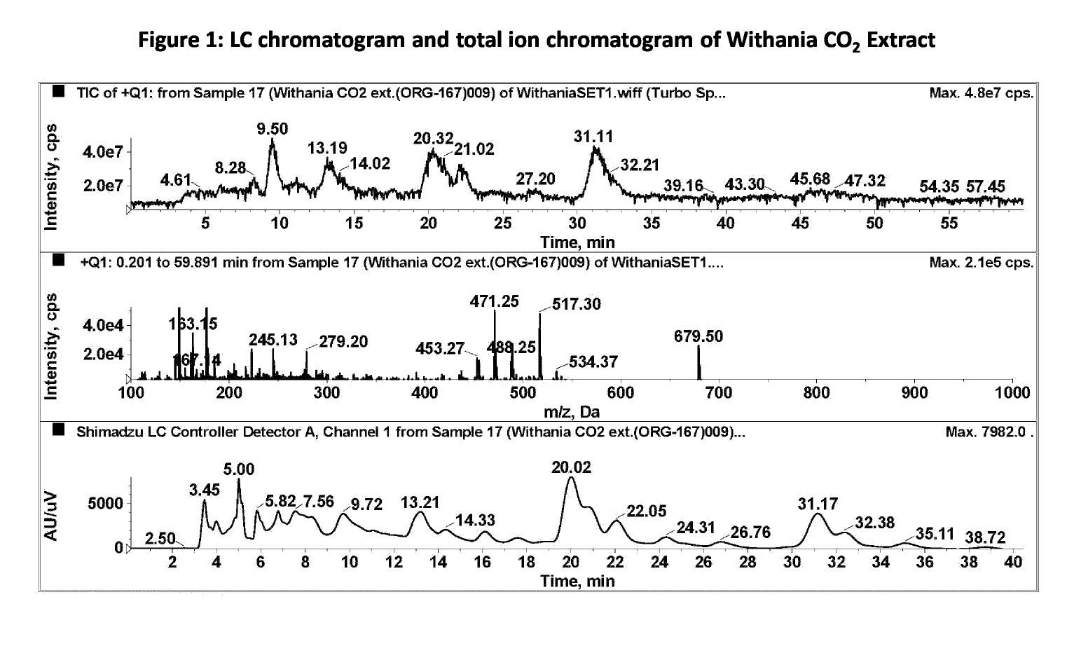

HPLC Analysis Method and Fingerprint of Supercritical Fluid (CO₂) Extract of Withania somnifera (FIG. 1)

LC-MS/MS (Applied Biosystems, API-2000) method of analysis for Withania CO₂ extract.
Conditions;
Column: RP C18 (250*4.6 mm, 5 um)
Flow rate: 0.5 ml/min
Run time: 45 min
Wave length: 223 nm, 254 nm.
Mobile phase: Methanol: Water (60:40)
Volume of injection: 20 ul
   Sample Preparation: (1 mg/ml Concentration)

Weighed accurately about 25 mg of Withania somnifera CO₂ extract in a 25 ml of clean volumetric flask. Added 20 ml of methanol (HPLC grade) and dissolved by sonication for 10 min. Make up to volume with methanol (HPLC grade). Filtered the final solution through 0.2 μm syringe filtered before injecting 20 μl to the instrument.

### EXAMPLE-10

Preparation of Zingiber officinale Water Extract

Approximately 100 Kg of shade dried plant material was subjected to extraction with 400 Liters of purified water by percolation method at Room Temperature. The water extractions after 24-48 hours were filtered through muslin cloth and concentrated to thick paste. After achieving the desired total solid content, the soft extract was spray dried to a free flowing dry extract powder. The water extract was also prepared by hot soxhlation method.

### EXAMPLE-11

Preparation of Zingiber officinale SCFE (CO₂) Extract

Approximately 25 Kg of the rhizomes of Zingiber officinale was pulverized to fine powder and loaded in to extractor. Super Critical Carbon dioxide liquid was pumped in to the extractor at a pressure of 300 bar and 39° C. temperature for 2-3 hours. Extract was separated into the container at pressure of 40 bar and 20° C. temperature. The CO₂ super critical liquid was recycled from the extraction vessel. The resultant extract was analyzed for active markers of Zingiber officinale.

### EXAMPLE-12

Preparation of Organic Zingiber officinale Granules (Formula-1)

TABLE 7

| S. | | Weight per | Weight per |
|---|---|---|---|
| No | Name of the Material | Tablet in mg | Batch In kg |
| 1 | Zingiber officinale (water extract) | 233.00 | 23.30 |
| | (#40 mesh) | | |
| 2 | Zingiber officinale (supercritical | 30.00 | 3.00 |
| | fluid extract, CO2) | | |
| 3 | Zingiber officinale aerial parts | 145.00 | 14.50 |
| | (# 60 mesh) | | |
| 4 | Zingiber officinale rhizome | 335.00 | 33.50 |
| | (# 60 mesh) | | |

### EXAMPLE-13

### Formula of Lubrication of Granules

TABLE 8

| S. | | Weight per | Weight per |
|---|---|---|---|
| No. | Name of the material | Tablet in mg | Batch in Kg |
| 1 | Zingiber officinale Extract Granules | 743 | 74.30 |
| | (#16 mesh) | | |
| 2 | Zingiber officinale aerial parts | 39.00 | 3.90 |
| | (#60) powder | | |
| 3 | Zingiber officinale rhizome | 38.00 | 3.80 |
| | (# 60) powder | | |
| | Total | 820.00 | 82.00 |

### EXAMPLE-14

### DISPENSING OF RAW MATERIAL

1. Dispense the raw materials as per Batch record

### Dry Heat Sterilization

1. Transfer 14.5 kg and 3.9 kg of Zingiber officinale aerial parts and 33.5 and 3.80 kg of Zingiber officinale rhizome powder into trays and sterilize the material@160° C. for 60 mins.
2. Unload the sterilized material in to double lined polybags separately and keep in airtight containers.
3. Check the sample for LOD, BD and Microbiological Analysis as per Table-9

TABLE 9

| S. No | Parameter | Standard values |
|---|---|---|
| 1 | LOD, Zingiber officinale aerial parts | 2.0-4.0% w/w |
| | powder | |
| | LOD, Zingiber officinale rhizome parts | 2.0-4.0% w/w |
| | powder | |
| 2 | BD, Zingiber officinale aerial parts | 0.2-0.3 g/ml |
| | powder | |
| | BD, Zingiber officinale rhizome parts | 0.3-0.4 g/ml |
| | powder | |
| 3 | TVAC | NMT 5000 cfu/gm/ml |
| 4 | Fungal count | NMT 10 cfu/gm/ml |

### Sifting

1. Sift 23.30 kg of water extract of Zingiber officinale rhizome through #40
2. Sift 14.5 and 33.5 kg of Zingiber officinale aerial parts powder and Zingiber officinale rhizome powder through #60
3. Sift 3.9 and 3.8 kg of Zingiber officinale aerial parts powder and Zingiber officinale rhizome powder through #60 separately
4. Collect the above-sifted materials in separate duly labeled double lined polybags.
5. Record Quantity Sifted and the sieve integrity before and after sifting

### Preparation of Granulation Fluid

1. Transfer about 10 kg of Purified water into a clean Stainless Steel Vessel. Record the observations. Record deviations if any.

### Granulation

1. Charge 23.30 kg of water extract of Zingiber officinale rhizome and 14.5 kg and 33.5 kg of Zingiber officinale aerial parts powder and Zingiber officinale rhizome powder into the RMG, mix for about 5 minute.
2. Slowly add 3.0 kg of supercritical extract of Zingiber officinale rhizome and granulation fluid to the RMG containing the sifted water extract of Zingiber officinale rhizome and powders of aerial parts of Zingiber officinale and powders of rhizome over a period of about 3 minute, with medium speed.
3. Stop the mixer and scrape off the mass from the sides and bottom.
4. Continue mixing by operating the impeller at high speed with Chopper ON for about 3 minute.
5. Add additional quantity of granulation fluid, if required.
6. Discharge the mass from the RMG.
7. Mill the Wet Mass obtained in Multi mill fitted with 8 mm screen.

### Tray Drying

1. Dry the Wet mass obtained in tray Drier at about 60° C. to 70° C. for about 60 minutes.
2. Check the Moisture once every 30 minutes. (LOD Limit: 2 to 4% w/w) and record the details

### Sizing

1. Sift the dried granules using a Sifter fitted with sieve #16.
2. Collect the sifted granules in a clean double polylined HDPE container.
3. Mill the retains (oversize granules) obtained through a Multi Mill fitted with 1.5 mm screen with ‘Knives Forward’ direction
4. Pass the milled granules obtained through a Sifter fitted with #16 sieve. Collect the sized granules
5. Weigh the granules. And analysed as per standard value. Table-10

TABLE 10

| | Parameter | Standard values |
|---|---|---|
| 1 | LOD | 2.0-4.0% w/w |
| 2 | BD | 0.40-0.60 g/ml |
| 4 | Actives | As per Finished Product spec |
| 5 | TVAC | NMT 5000 cfu/gm |
| 6 | Fungal count | NMT 10 cfu/gm |

### Blending

1. Transfer the sized granules into the blending area.
2. Transfer the sifted herb powder lubricant into the blending area.
3. Load 3.9 kg and 3.8 kg of Zingiber officinale aerial powder and Zingiber officinale rhizome powder and the sized granules into the Double Cone blender.
4. Blend the ingredients for 6 minutes at 10-11 RPM.
5. Unload the blend in a clean Double poly-lined HDPE drums and affix duly filled status labels.

### Compression

1. Check the Temperature and Relative Humidity of the area and record.
2. Bring the drums containing the blend into the compression area.
3. Carry out the initial checks before starting the operation as specified in below Table-11

TABLE 11

| | Description |
|---|---|
| Punch Size | 18 × 9 mm caplet |
| Upper Punch | Plain |
| Lower Punch | Plain |

4. Check for Appearance, Av. Wt., Individual wt., Thickness, Hardness, Friability & DT of 6 tablets
5. Check appearance, thickness & hardness of tablets every 30 min
6. Check Friability and DT every 1-hour
7. Check Average weight of 20 tablets every 30 mins graphically
8. Collect the tablets in a double poly-lined, tightly closed, container. Weigh each container and enter the details.
9. Collect the Compressed tablets in Double polylined HDPE drums and record their weights.

### EXAMPLE-15

Finished product specification of Zingiber officinale per caplet. Table-12

TABLE 12

| Parameters | Standard Range |
|---|---|
| Theoretical Average weight | 820 mg |
| Weight uniformity | 820 mg ± 5% (779 mg to 861 mg) |
| Weight of 20 tablets | 16.4 g ± 3% (15.90 gm to 16.89 gm) |
| Tablet thickness | 4.0 to 6.0 mm |
| Tablet hardness | 2 to 6 Kg/cm² |
| Friability | NMT 1.0% W/W |
| Disintegration time | NMT 30 min |
| Total gingerols content | 6.0 mg |

Pack tablets immediately after compression.

### Packing

1. Pack 60 tablets in a HDPE 120 CC container and weigh them for appropriateness of number of tablets.

### EXAMPLE-16

### Estimation of Total Gingerols by HPLC METHOD

### HPLC Conditions:

Column Details: C8 (Make: Thermo, Part No.: 30305-254630)
Wave Length: 282 nm
Flow rate: 1 ml/min
Volumn of Injection: 20 μl
Mobile Phase: Methanol:water (65:35)
Run Time: About 45 minutes

### Preparation of Standards:

Preparation of standard Gingerols (2 mg/ml): Weigh about 100 mg of reference standard (containing 6-Gingerol, 8-Gingerol, 6-Shagoal and 10-Gingerol) in a 50 ml volumetric flask, and dissolve by sonication in methanol. Make up the volume with methanol.

Preparation of sample:

For powdered raw material/Granules (10 mg/ml): Weigh 500 mg of the finely powdered sample in a 50 ml Volumetric flask, Dissolve with methanol and sonicate it for 5 minutes. Make the volume up to mark with methanol.

For standardized extracts (2 mg/ml): Weigh about 100 mg of the sample in a 50 ml volumetric flask, and dissolve by sonication in methanol. Make the volume up to the mark with methanol.

### Chromatographic Procedure:

After the stabilization of the instrument with the mobile phase, inject 20 μl of working standard solution into the column of the HPLC instrument. Record the chromatogram for about 45 minutes. Then inject 20 μl of the sample solution in the similar manner and record the chromatogram.

Calculation: Calculate the % total Gingerols content in sample by using the following formula in Table-13

TABLE 13
$\frac{\begin{matrix}Total AUC of the peaks in the sample \\ chromatogram corresponding to peaks in \\ standard\end{matrix}}{\begin{matrix}AUC of the peaks in the standard \\ chromatogram\end{matrix}} \times \frac{\begin{matrix}Concentration \\ of standard \\ ( mg \text{/} ml )\end{matrix}}{\begin{matrix}Concentration \\ of sample \\ ( mg \text{/} ml )\end{matrix}} \times \begin{matrix}Purity \\ of \\ standard\end{matrix}$

### EXAMPLE-17

Liquid Chromatography—Mass Spectrometer Analysis of Zingiber officinale Supercritical (CO2) Extract and Water Extract:

LCMSMS analysis were carried out by using an applied biosystem—Sciex API 2000 triple quadrupole mass spectrometer equipped with an atmospheric pressure chemical ionization source and heated nebulizer APCI interface. The liquid chromatography was a LC-20 AD Series binary system equipped with an autosampler. The column used was C18 phenomenex (250×4.6 mm, 5 μm), flow rate 1 ml/min of mobile phase methanol:water (65:35), wave length 282 nm and run time 40 min. The analytes were ionized by APCI in positive-ion mode (PI mode). Final ionization conditions were heated nebulizer temperature 450° C., curtain gas Nitrogen 30 psi, particulate-free and CO2-free air was used as nebulising gas at a flow rate of 70 psi.

### Sample Preparation for Ginger CO2 Extract:

Weigh about 50 mg of sample in a 50 ml volumetric flask, and dissolve by sonication in methanol (HPLC grade). Make the volume up to the mark with methanol filter through 0.2 um syringe filter.

### Sample Preparation for Ginger Water Extract:

Weigh about 500 mg of finely powdered sample in a 50 ml volumetric flask, and dissolve by sonication in methanol (HPLC grade). Make the volume up to the mark with methanol filter through 0.2 um syringe filter

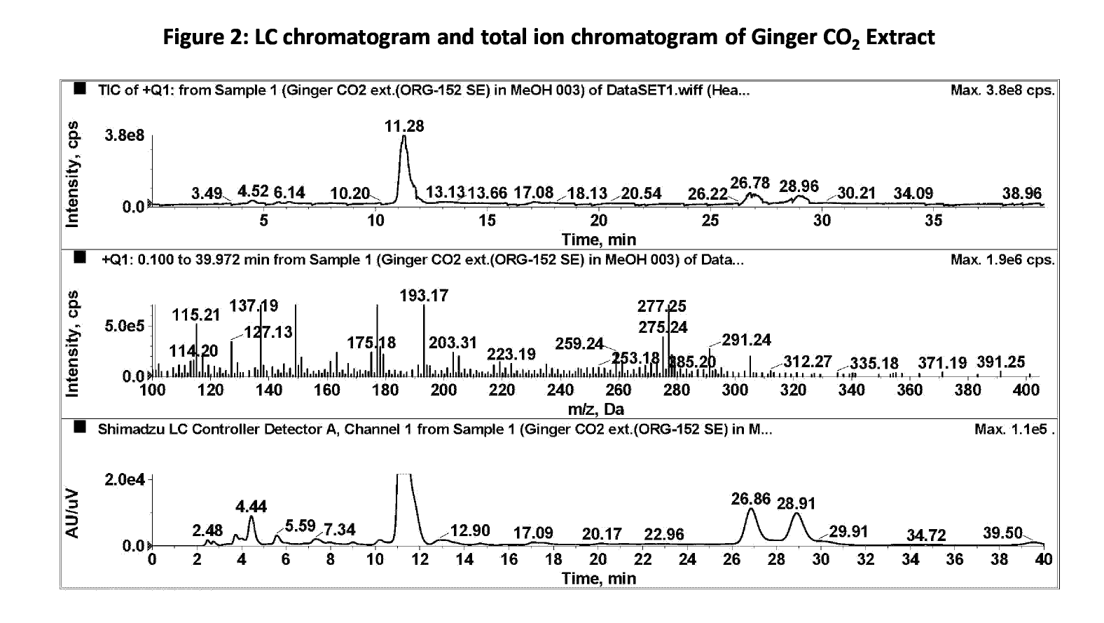

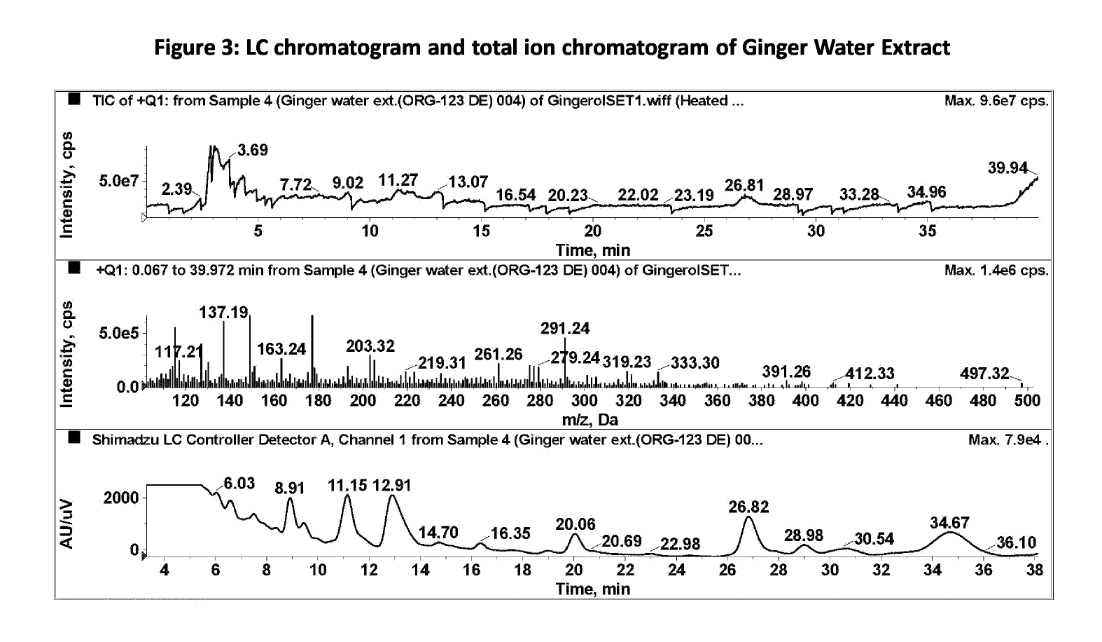

LCMSMS chromatogram is represented in FIG. 2 and 3.

### EXAMPLE-18

Preparation of Commiphora mukul Water Extract

Approximately 100 Kg of dried herbal resin was subjected to extraction with 400 Liters of purified water by percolation method at Room Temperature. The water extractions after 24-48 hours were filtered through muslin cloth and concentrated to thick paste. After achieving the desired total solid content, the soft extract was spray dried to a free flowing dry extract powder. The water extract was also prepared by hot soxhlation method.

### EXAMPLE-19

Preparation of Commiphora mukul SCFE, Super Critical Fluid Extract (CO₂) Extract

Approximately 25 Kg of resin of Commiphora mukul was pulverized to fine powder and loaded in to extractor. Super Critical Carbon dioxide liquid was pumped in to the extractor at a pressure of 300 bar and 39° C. temperature for 2-3 hours. Extract was separated into the container at pressure of 40 bar and 20° C. temperature. The CO₂ super critical liquid was recycled from the extraction vessel. The resultant extract was analyzed for active markers of Commiphora mukul.

### EXAMPLE-20

### Formula of Granulation

TABLE 14

| | | Weight per | Weight per |
|---|---|---|---|
| S. | | capsule | batch |
| No | Raw Materials | in mg | in kgs |
| 1 | Commiphora mukul water extract | 195.00 | 19.50 |
| | (40 #) | | |
| 2 | Commiphora mukul supercritical fluid | 60.00 | 6.00 |
| | extract (SCFE) CO₂ Extract | | |
| 3 | Commiphora mukul stem powder | 75.00 | 7.50 |
| | (80 #) | | |
| 4 | Purified water | Quantity | |
| | | sufficient | |

### Formula for Lubrication

TABLE 15

| | | | Weight | |
|---|---|---|---|---|
| | | | per | Weight |
| S. | | | capsule | per batch |
| No. | Raw materials | Name of material | in mg/caps | in kg |
| 1 | Commiphora mukul | Commiphora | 330.00 | 33.00 |
| | granules (16 #) | mukul granules | | |
| 2 | Commiphora mukul | Commiphora | 30.00 | 3.00 |
| | stem powder (80 #) | mukul stem | | |
| | | powder (80 #) | | |
| 3 | E veg capsules CT/CT | Empty veg | — | 100000 |
| | “0” | capsules | | |
| | Fill weight | | 360.00 | 36.00 |

### EXAMPLE-21

### Manufacturing Process of Granules and Capsules

### Dispensing of Raw Material

1. Dispense the raw materials as per Batch Formula

### Dry Heat Sterilization

1. Transfer 10.5 kg of Commiphora mukul stem powder in to trays and sterilize the material@160° C. for 60 mins.
2. Unload the sterilized materials in to double lined polybags separately and keep in airtight containers.
3. Send the sample for Loss on drying, Bulk density and Microbiological Analysis. The parameters are mentioned in below table-16

TABLE 16

| | Parameter | Standard values |
|---|---|---|
| 1 | LOD | 2.5-3.5% |
| 2 | BD | 0.15-0.25 g/ml |
| 3 | Microbes | NMT 5000 cfu/gm |
| 4 | Fungal count | NMT 10 cfu/gm |

### Sifting

1. Sift Commiphora mukul water extract through #40 and Commiphora mukul stem powder through #80, weigh as per the required quantity and kept separately in duly-labeled double lined poly bag.
2. Record Quantity Sifted and the sieve integrity before and after sifting.

### Preparation of Granulation Fluid

1. Transfer about 15.0 kg of Purified water into a clean Stainless Steel Vessel
2. Take 6.00 liters of purified water in a SS vessel, add 3.5 kg of Commiphora mukul stem powder then mix continuously to get a uniform solution.

### Granulation

1. Charge 16.00 Kg of Commiphora mukul water extract, 7.50 kg of Commiphora mukul stem powder into the RMG, mix for 5 minute.
2. Add slowly 6 kg of Commiphora mukul SCFE extract to the above blend. Mix it for 5 minutes.
3. Add step 7.2 to RMG and granulate. If required add additional quantity of purified water, mix over a period of 3 minute, with medium speed.
4. Stop the mixer and scrape off the mass from the sides and bottom.
5. Continue mixing by operating the impeller at high speed with Chopper ON for 3 minute.
6. Add additional quantity of purified water, if required.
7. Discharge the mass from the RMG.
8. Record the observations. Record deviations if any.

### Wet Milling

1. Mill the Wet Mass obtained in Multi mill fitted with 8 mm screen.
2. Record the observations.
3. Record deviations if any.

### Tray Drying

1. Dry the Wet mass obtained in tray Drier at about 60° C. for about 60 minutes.
2. Check the Moisture once every 30 minutes. (LOD Limit: 2-4%) and record the details

### Sizing

1. Sift the dried granules using a Sifter fitted with sieve #16.
2. Collect the sifted granules in a clean double poly-lined HDPE container.
3. Mill the retains (oversize granules) obtained through a Multi Mill fitted with 1.5 mm screen with ‘Knives Forward’ direction
4. Pass the milled granules obtained through a Sifter fitted with #16 sieve.
5. Sizing of dried granules must be carried out within 2 hours. In case the dried granules are not sized immediately, collect them in double poly-lined HDPE container, duly labeled.
6. Collect the sized granules obtained and add them to the sifted granules

### Blending

The granules were evaluated for following parameters (Table-17).

TABLE 17

| | Parameter | Standard values |
|---|---|---|
| 1 | LOD | 2-4% |
| 2 | BD | 0.45-0.55 g/ml |
| 3 | Granules to fine ratio | 60:40 to 70:30 |
| 4 | Actives | As per Finished Product spec |
| 5 | Microbes | NMT 5000 cfu/gm |
| 6 | Fungal count | NMT 10 cfu/gm |

1. Maintain the Temperature and Relative Humidity of the area with in the specified limit (Limit Temperature NMT 25° C. and relative humidity NMT 40%)
2. Transfer the sized granules into the blending area.
3. Transfer the sifted Commiphora mukul stem powder lubricant into the blending area.
4. Load required quantity of Commiphora mukul stem powder and the sized granules into the Double Cone blender.
5. Blend the ingredients for 3 minutes at 20-25 RPM.
6. Record the details
7. Unload the blend in a clean Double poly-lined HDPE drums and affix duly filled status labels.
8. Weigh the blend and enter the details.
9. Record deviations if any.

### Capsule Filling

1. Maintain the Temperature and Relative Humidity of the area with in the specified limit.
2. (Limit Temperature NMT 25° C. and relative humidity NMT 40%).
3. Check the Temperature and Relative Humidity of the area and record.
4. Bring the drums containing the blend into the capsule filling area.
5. Adjust the machine as per the parameters mentioned in stage 13.11.
6. Carry out the initial checks before starting the operation.
7. Check for Appearance, Av. Wt., Individual wt., length & DT of 6 capsules.
8. Check for the appearance, length of capsules every 30 min.
9. Check DT every 1-hour.
10. Check Av. weight of 20 capsules every 30 mins graphically.
11. Collect the capsules in a double poly-lined, tightly closed, container. Weigh each container and enter the details.
12. In process specification for capsules (Table-18)

TABLE 18

| Parameter | Standard limit |
|---|---|
| Description | Clear Transparent size 0 capsules filled |
| | with brown colored granules |
| Weight of empty capsules | 95-100 mg |
| Fill weight | 360 mg |
| Average weight | 460 ± 5% |
| Weight 20 capsules | 9.2 gm ± 3% |
| Average length of 10 capsules | 21.4 ± 0.4 mm |
| DT | NMT 15 min |
| Guggul sterones | NLT 3.75 mg/capsule |

1. Collect the filled capsules in double poly-lined HDPE drums and record their weights.
2. Affix duly filled status labels to the containers.
3. Perform the reconciliation of the filled capsules.

### Packing

1. Pack 60 capsules in a HDPE 120 CC container and weigh them for appropriateness of number of capsules.
2. Put about 15 grams of cotton with minimum pressing and make sure that rattling is minimum.

### EXAMPLE-22

### Formula of Granulation for Commiphora mukul and Piper longum

TABLE 19
Granulation and lubrication formula

| | | Weight per | |
|---|---|---|---|
| S. | | capsule | Weight per |
| No | Raw Materials | in mg | batch in kgs |
| 1 | Commiphora mukul water extract (40 #) | 180.00 | 0.900 |
| 2 | Commiphora mukul supercritical fluid | 60.00 | 0.300 |
| | extract (SCFE) CO₂ Extract | | |
| 3 | Commiphora mukul stem powder (80 #) | 250.00 | 1.250 |
| 4 | Piper longum seed powder (80 #) | 260.00 | 1.300 |
| 5 | Purified water | Quantity | 2.400 litres |
| | | sufficient | |

### EXAMPLE-23

### Processing Details

### Sifting

1. Sift Commiphora mukul water extract through #40 mesh, Commiphora mukul herb powder 80# and Piper longum seed powder #80.

### Granulation

1. Transfer about 3.0 Ltr of Purified water and heat to 80-90° C. for 30 min into a clean Stainless
2. Steel Vessel. Use this boiled cooled water for the granulation.
3. Charge Commiphora mukul (Guggul) water extract, Commiphora mukul (Guggul) herb powder, Piper longum seed powder into the RMG and dry mix for about 2 minute, and then add Commiphora mukul CO₂ extract into the RMG, mix for about 3 minute.
4. Slowly add the granulation fluid to the above blend and granulate for about 3 minute. Stop the mixer and scrape off the mass from the sides and bottom.
5. Continue mixing by operating the impeller at high speed with Chopper ON for about 1 minute. Add additional quantity of granulation fluid, if required.
6. Discharge the mass from the RMG.

### Wet Milling

1. Mill the wet mass in multi mill fitted with 8 mm screen.

### Drying

1. Dry the Wet mass obtained in fluid bed drier at about 50° C. to 60° C. for about 60 minutes.
2. Check the Moisture. (LOD Limit: 2.0 to 3.0%) and record the details.

### Sizing

1. Sift the dried granules using a Vibratory sifter fitted with sieve #16.
2. Collect the sifted granules in a clean double polylined HDPE container.
3. Mill retains the oversized granules through a Multi Mill fitted with 1.5 mm screen.
4. ‘Knives Forward’ direction Pass the milled granules through a Sifter fitted with #16 sieve.

### Blending

Blend the granules for about 5 minute at 10-15 RPM in octagonal blender.

### Compression

1. Compress the granules into 17×8 mm plain caplet shaped punch.
2. Carry out the compression and check for the standard parameters (Table-20).

TABLE 20
STANDARD PARAMETERS

| | |
|---|---|
| 1. Average weight | 750 mg |
| 2. Weight uniformity | 750 mg ± 5% (712.5 mg to 787.5 mg) |
| 3. Weight of 20 caplets | 15.00 g ± 3% (14.55 gm to 15.45 gm) |
| 4. Tablet hardness | NLT 4.0 Kg/cm² |
| 5. Friability | NMT 1.0% W/W |
| 6. Disintegration time | NMT 30 min. |

### EXAMPLE-24

FORMULA—Commiphora mukul (Guggul) Tablets (Round) Table-21

TABLE 21

| S. | | | |
|---|---|---|---|
| NO | NAME OF THE INGREDIENT | MG PER | QTY/BATCH IN |
| Granulation formula | | DOSAGE | KG 10000 caplets |
| 1 | Commiphora mukul water extract | 250.00 | 2.50 |
| | #40 | | |
| 2 | Commiphora mukul stem | 250.00 | 2.50 |
| | powder #40 | | |
| 3 | Purified water | Quantity | 1.0 Ltr |
| | | sufficient | |
| 4 | Commiphora mukul granules #16 | 500.00 | 5.000 |

### Blending Formula Table-22

TABLE 22

| | | | Weight per | |
|---|---|---|---|---|
| S. | | Name of | capsule | Weight per |
| No. | Raw materials | material | in mg/caps | batch in kg |
| 1 | Commiphora mukul | Commiphora | 500.00 | 50.00 |
| | granules (16 #) | mukul granules | | |
| 2 | Commiphora mukul | Commiphora | 100.00 | 10.00 |
| | stem powder (40 #) | mukul stem | | |
| | | powder (40 #) | | |
| | Fill weight | | 600.00 | 60.00 |

### Processing Details:

### Sifting

1. Sift Commiphora mukul water extract and Commiphora mukul stem powder through #40 mesh.

### Preparation of Granulation Fluid

1. Transfer about 1.0 Ltr of Purified water and heat to 80-90° C. for 30 min into a clean Stainless
2. Steel Vessel. And use boiled cooled water for the granulation.

### Granulation

1. Charge Commiphora mukul water extract, Commiphora mukul stem powder in to the Rapid mixture granulator and dry mix for about 2 minutes, Slowly add the granulation fluid to the above blend and granulate for about 5 minutes.
2. Stop the mixer and scrape off the mass from the sides and bottom. Continue mixing by operating the impeller at high speed with Chopper ON for about 1 minute.
3. Add additional quantity of granulation fluid, if required. Discharge the mass from the rapid mixture granulator.

### Wet Milling

1. Mill the wet mass in multi mill fitted with 8 mm screen.

### Drying

1. Dry the Wet mass obtained in fluidized bed dryer FBD at about 50° C. to 60° C. for about 60 minutes.
2. Check the Moisture. (LOD Limit: 2.0 to 3.0%) and enter the details.

### Sizing

1. Sift the dried granules using a Vibratory sifter fitted with sieve #16.
2. Collect the sifted granules in a clean double polylined HDPE container.
3. Mill the retains the oversized granules through a Multi Mill fitted with 1.5 mm screen in ‘Knives Forward’ direction Pass the milled granules through a Sifter fitted with #16 sieve.

### Blending

1. Blend granules with Commiphora mukul stem powder for about 5 minute at 10-15 RPM in octagonal blender OGB.

### Compression

1. Compress the granules in to 12.6 mm 40 through round shaped punch.
2. Carry out the compression and check for the standard parameters, (Table-23)

TABLE 23
STANDARD PARAMETERS

| | |
|---|---|
| 1. Average weight | 600 mg |
| 2. Weight uniformity | 600 mg ± 5% (570.0 mg to 630.0 mg) |
| 3. Weight of 20 caplets | 12.00 g ± 3% (11.64 gm to 12.36 gm) |
| 4. Tablet hardness | NLT 4.0 Kg/cm² |
| 5. Friability | NMT 1.0% W/W |
| 6. Disintegration time | NMT 30 min. |

### EXAMPLE-25

1. Estimation of guggul sterones by HPLC Standard preparation (1 mg/ml): Weigh accurately abot 50 mg of standard Guggul sterone in a 50 ml volumetric flask. Add 40 ml of acetonitrile and dissolve by sonication. Make the volume up to the mark with acetonitrile.
2. Sample preparation: Weigh accurately about 50 mg of sample in a 50 ml volumetric flask. Add 40 ml of acetonitrile and dissolve by sonication. Make the volume up to the mark with acetonitrile.

HPLC Conditions are as follows.
Column: C₁₈ hypersil ODS column with dimension 250×4.6 mm, particle size—5μ
Solvent system: Acetonitrile:Water (60:40)
Flow rate: 1 ml/min
Detection: 241 nm
Injection volume: 20 μl
Run time: Approximately 30 minutes
Approximate retention time: Guggul sterone E-10 minutes, Guggul sterone Z-13 minutes.

Chromatographic procedure: Inject 20 μl of standard to the HPLC injector and record the chromatogram. Two major peaks obtained in the chromatogram correspond to Guggul sterone E and Z isomers. In the similar manner inject 20 μl of sample and record the chromatogram. Add the AUC of both the major peaks (Isomers E and Z) for the purpose of calculation.
Calculation: Table-24

TABLE 24
$\begin{matrix}\% \\ Guggul \\ sterone\end{matrix} = \frac{AUC of sample}{AUC of standard} \times \frac{Concentration of standard}{\begin{matrix}— \\ Concentration of sample\end{matrix}} \times \begin{matrix}\% Purity \\ of \\ standard\end{matrix}$

### EXAMPLE-26

Details on Amino Acid Composition in Water Extract of Commiphora mukul.

Analysis method: Journal of Association of Official Agricultural Chemists, 70, 241-247, 1987. Results are summarized in Table-25.

TABLE 25

| S. No. | Amino Acid | % in water extract |
|---|---|---|
| 1 | Asparagine | 1.69 |
| 2 | Glutamine | 1.69 |
| 3 | Serine | 1.56 |
| 4 | Glycine | 1.28 |
| 5 | Histidine | 0.33 |
| 6 | Arginine | 0.52 |
| 7 | Threonine | 1.16 |
| 8 | Alanine | 1.45 |
| 9 | Proline | 1.24 |
| 10 | Tyrosine | 1.16 |
| 11 | Valine | 1.16 |
| 12 | Methionine (estimated as methionine | 0.24 |
| | sulfone) | |
| 13 | Cystine (estimated as cysteic acid) | 0.52 |
| 14 | Isoleucine | 1.39 |
| 15 | Leucine | 2.25 |
| 16 | Phenylalanine | 1.07 |
| 17 | Lysine | 0.93 |

### EXAMPLE-27

### LCMSMS Analysis of Commiphora mukul Extract (FIG. 1)

LCMSMS method of analysis for Commiphora mukul, super critical fluid extract, CO₂ extract.
Instruments details: LC-MS/MS (Applied Biosystems, API-2000)

### Conditions:

Column: RP C18 (250*4.6 mm, 5 um)
Flow rate: 0.5 ml/min
Run time: 60 min
Wave length: 241 nm, 254 nm.
Mobile phase: Acetonitrile:Water (60:40)
Volume of injection: 20 ul
   Sample Preparation: (1 mg/ml)

Weigh accurately about 25 mg of Commiphora mukul CO₂ extract in a 25 ml of clean volumetric flask. Add 20 ml of Acetonitrile (HPLC grade) and dissolve by sonication for 10 min. Make up to volume with Acetonitrile (HPLC grade). Filter the final solution through 0.2 μm syringe filter before injecting 20 μl to the instrument.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

Please find abbreviated terms for following.
1) LOD—Loss on drying
2) BD—Bulk density
3) RMG—Rapid Mixer Granulator
4) FBD—Fluid Bed Drier
5) TVAC—Total Viable Aerobic Count
6) NMT—Not More Than
7) DT—Disintegration Time

We claim:

1. A herbal solid formulation comprising a blend of Super Critical Fluid (CO₂) extract, water extract and powder of herb Withania somnifera, Zingiber officinale or Commiphora mukul, wherein said blend of extract and said powder of herb is mixed in a ratio of about 1:0.5 to about 1:10.

2. The herbal solid formulation of claim 1, wherein said herbal solid formulation is essentially free of additives/excipients.

3. The process of claim 1, wherein the extract/powder is obtained using resin & stem of Commiphora mukul, resin & stem of Withania somnifera and bulb & aerial of Zingiber officinale.

4. The herbal solid formulation of claim 1, wherein said solid formulation is preferably granules, tablet or capsule.

5. The herbal solid formulation of claim 1, wherein said solid formulation is a tablet.

6. The herbal solid formulation of claim 5, wherein the tablet is having hardness of about 3 to about 4 kg/cm², a friability of less than about 1% and disintegration time is less than about 30 min.

7. The herbal solid formulation of claim 5, wherein the tablet is having disintegration time is less than about 10 min.

8. A process for preparing a herbal solid formulation as claimed in claim 1, comprising:
autoclaving powder of a herb;
granulating the autoclaved powder with a water extract of the herb;
lubricating the granulated mixture by adding the autoclaved powder of the herb; and
preparing the solid formulation.

9. The process of claim 8, wherein the extract of herb is obtained by employing Super Critical Fluid (CO₂) extraction, percolation, hot soxhalation or refluxing.

10. The process of claim 9, wherein the percolation, hot soxhalation or refluxing method is performed in the presence of a solvent selected from water, grain alcohol or combinations thereof.

11. The process of claim 8, wherein the powder of herb is obtained by pulversing the herb to a powder having mesh size between about 10 to about 100.

12. The process of claim 11, wherein the powder of herb is obtained by pulversing the herb to a powder having mesh size between about 20 to 80.

13. The process of claim 8, wherein the granulation is carried out by employing a solvent system selected from water, grain alcohol or combinations thereof.
